(19) United States
(12) Patent Application Publication
(10) Pub. No.: US 2007/0225759 A1
(43) Pub. Date: Sep. 27, 2007

(54) Method for delivering a medical device to the heart of a patient

(76) Inventors: Daniel Thommen, Steinhausen (CH); Jerome Bernhard, Zurich (CH)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/387,195

(22) Filed: Mar. 22, 2006

(51) Int. Cl. A61B 17/08

(52) U.S. Cl. 606/213

(57) ABSTRACT

A method is provided for delivering a medical device to the heart of a patient including the steps of providing a medical device such as an occluder; providing a delivery equipment for delivering the medical device to the heart; performing a transthoracic puncture; introducing the delivery equipment and the device through the puncture to a location in the heart; implanting the device within the location; detaching the device from the delivery equipment and removing the delivery equipment from the heart of the patient. This method enables a correction of the ventricular septal defect.

### FIELD OF THE INVENTION

The invention relates to a method for delivering a medical device to the heart of a patient. In one preferred variant, this invention relates to a method for delivering an occluder to the heart of a patient.

### BACKGROUND OF THE INVENTION

Medical devices are well known in percutaneous cardiac surgery and in aorta surgery. These devices include stents, valves and occluders. Such medical devices are usually delivered percutaneously through an intravascular sheath into a patient. This enables the physician to deliver and position the device within the human body in a minimally invasive manner.

Such occluders are for example disclosed in U.S. Pat. No. 6,488,706, U.S. Pat. No. 6,949,116 and WO 2005/074813, which are all assigned to the assignee of the present invention. These disclosures are incorporated in this application by reference.

The method mentioned above works well when the occluder is used for permanent occlusion of an atrial septal defect. However, the percutaneous delivery of a device to the interventricular septum, especially for occluding a ventricular septal defect, is more difficult. There are two possibilities known in the state of the art to deliver a device to this place:
a) through the inferior vena cava into the right atrium, then with a transseptal puncture into the left atrium and then through the mitral valve into the left ventricle or
b) through the aorta and the mitral valve to the left ventricle.

The first method, which is called transseptal puncture, has the disadvantage that the sheath and the device have to be moved along a very winding way. The risk to injure the surrounding tissue is quite high. The second method, which is called retrograde access, has the disadvantage that the occluder has to be introduced against the circulatory flow of the blood.

There is a demand therefore, for a method of introducing or delivering an occluder into the interventricular septum or similar placement which avoids or eliminates these disadvantages. The present invention satisfies the demand.

### SUMMARY OF THE INVENTION

It is therefore an object of the invention to provide a new method for delivering a medical device, especially an occluder, to the heart of a patient. This method is especially successful when the device is delivered to the interventricular septum.

A method according to one embodiment of the invention includes the steps of:
a. providing a medical device;
b. providing a delivery equipment for delivering the medical device to the heart;
c. performing a transthoracic puncture;
d. introducing the delivery equipment and the device through the puncture to a location in the heart;
e. placing the device at the location;
f. detaching the device from the delivery equipment and
g. removing the delivery equipment from the heart of the patient.

It is another object of the invention to provide a new method for delivering a medical device into a body of a patient.

This method according to another embodiment of the invention includes the steps of:
a. providing a medical device;
b. providing a delivery equipment for delivering the medical device into the body;
c. performing a transthoracic puncture;
d. introducing the delivery equipment and the device through the puncture to the heart;
e. forwarding the delivery equipment and the device through the heart to a location of treatment;
f. detaching the device from the delivery equipment and
g. removing the delivery equipment from the heart and the body of the patient.

The location can be a defect or just a location which needs special treatment. In the first method mentioned above, the location is in the heart or at an edge of the heart. In the second method mentioned above the location is outside or at an edge of the heart. This location can for example be in the aorta.

The placement of the device at the location can be an implantation, a fixation of the device or another treatment at the location. The patient is preferably human but it can also be an animal, especially a mammal.

Especially when a ventricular septal defect should be corrected the transthoracic puncture is preferably performed just beside the sternum of the patient. This access angle guarantees a good access to the interventricular septum. Using this angle a needle used for the transthoracic puncture can be pushed via the right ventricle and the interventricular septum into the left ventricle of the heart.

Preferably the device is an implant, especially an occluder. Preferably it is an occluder as described or claimed in the patent publications as mentioned above. However the invention is not restricted to occluders but can also be performed with stents, valves or other known devices.

The delivery equipment includes usually a sheath or a catheter and some means to place and fix the device. The means depend on the device used and are known in the state of the art.

Further advantageous variants can be gathered from the dependent claims.

### BRIEF DESCRIPTION OF THE DRAWINGS

The subject matter of the invention is explained hereinbelow with reference to preferred exemplary embodiments illustrated in the attached drawings.

FIG. 1 shows a schematic cross section of a human heart, with an occluder placed to occlude a ventricular septal defect. The figure shows on the right hand side the occluder in a position during the delivering procedure and on the left hand side the occluder in its final position and shape and

FIG. 2 shows a schematic cross section of a human heart with two medical devices placed within the heart.

### DETAILED DESCRIPTION OF EMBODIMENTS OF THE INVENTION

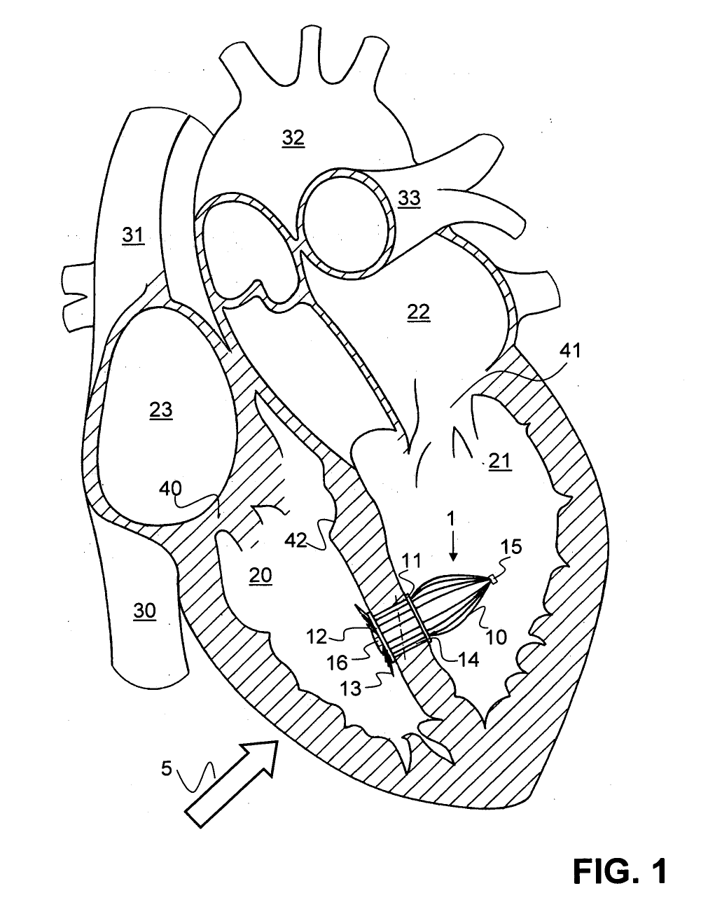

FIG. 1 shows a schematic cross section of a human heart. The inventive method is preferably performed with a human heart. However it is not restricted to human surgery but can also be used for delivering devices into animal hearts.

The relevant parts of the human heart are mentioned below, however, the full medical details are well known in the state of the art. The heart includes a right ventricle 20, a left ventricle 21, a left atrium 22 and a right atrium 23. Between the left ventricle 21 and the left atrium 22 the mitral valve 41 is arranged, and between the right ventricle 20 and the right atrium 23 the tricuspid valve 40 can be found. The right and the left ventricle 20, 21 are separated by the interventricular septum 42. Conduits to and from the heart respectively are the inferior vena cava 30, the superior vena cava 31, the aorta 32 and the pulmonary artery 33.

A ventricular septal defect is a defect, i.e. a hole, in the interventricular septum. This defect is well known in the state of the art. This hole can be closed by an occluder as shown in FIG. 1.

Preferably, an occluder as described in WO 2005/074813 is used. This occluder comprises a plurality of thin elongate members 10, 13, 14, such as wires or threads, a first occluding body or membrane 11 and a second occluding body or membrane 12, a first holder 15 and a second holder 15. The occluder is compressed into a sheath when introduced into the heart and placed within the defect in the interventricular septum 42.

On the right hand side of the figure, the occluder 1 is shown in the non-compressed, but still not fixed, state. On the left hand side of the figure, the occluder 1 is shown in the fixed state where the elongate members 12 are twisted into a flowerlike shape.

The equipment used for placing and opening the occluder 1 at the location of the defect is also described in the patent publications mentioned earlier in this text. The equipment includes a sheath and a pushing rod to move the two holders to each other and to fix them at the location of the defect.

The situation shown on the left and right hand side may not occur at the same moment. In practice, the elongate members 10 on the right hand side do actually twist first, i.e. before the ones on the other side, or they may twist at the same time as the one on the other side.

It is also possible to use an occluder which comprises only one single membrane as shown in U.S. Pat. No. 6,488,706 or U.S. Pat. No. 6,949,116. However the inventive method can also be performed with other occluders known in the state of the art. Also other medical devices, for example stents or valves, can be used with the same inventive method. Such medical devices are for example described in U.S. Pat. No. 5,928,280, U.S. 2005/0228434 and U.S. Pat. No. 5,352,240.

According to embodiments of the inventive method, direct transthoracic puncture is used. With this method the heart is directly accessed percutaneously through the anterior chest wall. The preferred direction used is shown in the figure with an arrow bearing the reference number 5. Using the delivery equipment described in the patent publications mentioned above or with other suitable equipment known in the state of the art, the device is delivered to the heart. Preferably a sheath is used for delivering and placing the device. Depending on the kind of device the sheath or another tool can be used to open the device at the location of the defect, after the device is placed correctly within the defect.

Two animal tests performed on pigs are reported below:

In the first test, a direct transthoracic puncture of the right ventricle was performed with a needle. With the Seldinger technique, the needle was exchanged to a standard introducer.

In the second test, a transthoracic puncture was performed on the right side of the sternum. The angle allowed the pushing of the needle via the right ventricle and the septum into the left ventricle. After insertion of a 10 F introducer a hole was dilated in the interventricular septum with a dilation balloon, 12 mm in diameter. Through the same introducer a 15 mm occlusion device was placed in the septal defect.

The inventive method therefore allows deliverance of a device to parts of the heart which can not be accessed so easily. This method is suitable for delivering an occluder to correct a ventricular septal defect. However, this method can also be used to occlude other parts of the heart. Furthermore, this method can also be used for delivering stents, valves or other cardiac or intracardiac devices.

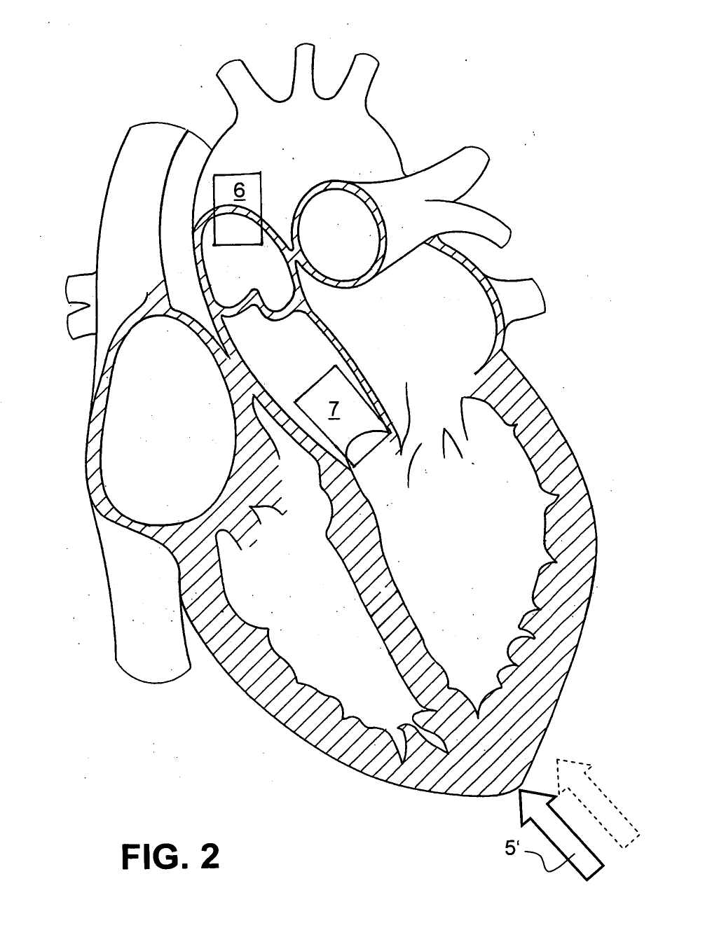

FIG. 2 shows a human heart with two medical devices located in the heart. These devices as well were introduced into the heart. The devices are only shown schematically as a rectangle, however they can have any appropriate shape known in the state of the art. Reference number 6 concerns a stent; reference number 7 concerns a valve. The preferred direction is shown in the figure bearing the reference number 5′. The place of introduction into the heart can be at the apex of the heart (arrow with straight lines) or just beside the apex (arrow with dashed lines).

The medical devices can also be placed outside or at the edge of the heart wherein the way of delivery always goes through the heart.

The described embodiments are to be considered in all respects only as illustrative and not restrictive, and the scope of the invention is, therefore, indicated by the appended claims rather than by the foregoing description. Those of skill in the art will recognize changes, substitutions and other modifications that will nonetheless come within the scope of the invention and range of the claims.

What is claimed is:

1. A method for delivering a medical device to the heart of a patient, the method comprising the steps of:
a. providing a medical device;
b. providing a delivery equipment for delivering the medical device to the heart;
c. performing a transthoracic puncture;
d. introducing the delivery equipment and the device through the puncture to a location in the heart;
e. placing the device at the location;
f. detaching the device from the delivery equipment, and
g. removing the delivery equipment from the heart of the patient.

2. The method according to claim 1 wherein said transthoracic puncture is performed just beside the sternum of the patient.

3. The method according to claim 1 wherein a needle is used for said transthoracic puncture which needle is pushed via a right ventricle and a septum of the heart into a left ventricle of the heart.

4. The method according to claim 1 used to correct a ventricular septal defect.

5. The method according to claim 1 wherein said device is an occluder.

6. The method according to claim 5 wherein said occluder is an occluder from the group claimed in U.S. Pat. No. 6,488,706, U.S. Pat. No. 6,949,116 and WO 2005/074813.

7. The method according to claim 1 wherein a sheath is used for delivering and placing said device.

8. The method according to claim 1 wherein said device is opened at said location of the defect.

9. A method for delivering a medical device into a body of a patient, the method comprising the steps of:
a. providing a medical device;
b. providing a delivery equipment for delivering the medical device into the body;
c. performing a transthoracic puncture;
d. introducing the delivery equipment and the device through the puncture to the heart;
e. forwarding the delivery equipment and the device through the heart to a location of treatment;
f. detaching the device from the delivery equipment, and
g. removing the delivery equipment from the heart and the body of the patient.

10. The method according to claim 9, wherein the medical device is forwarded to a location within the aorta.
